(19) United States
(12) Patent Application Publication
(10) Pub. No.: US 2011/0256118 A1
(43) Pub. Date: Oct. 20, 2011

(54) ANTIOXIDANT-CONTAINING FOOD COMPOSITION FOR USE IN ENHANCING ANTIVIRAL IMMUNITY IN COMPANION ANIMALS

(75) Inventors: Jeffrey A. Brockman, Lawrence, KS (US); Nolan Zebulon Frantz, Meadville, PA (US); Steven C. Zicker, Lawrence, KS (US)

(73) Assignee: Hill's Pet Nutrition, Inc., Topeka, KS (US)

(21) Appl. No.: 13/140,143

(22) PCT Filed: Dec. 16, 2009
(86) PCT No.: PCT/US09/68230

§ 371 Date: Jun. 16, 2011

Related U.S. Application Data
(62) Division of application No. 61/122,920, filed on Dec. 16, 2008.

(51) Int. Cl. A61K 31/385; A61K 31/7076; A61P 31/12; A61K 36/16; A61P 3/02; A61K 33/04; A61K 38/06

(52) U.S. Cl. 424/94.1; 514/440; 424/702; 514/46; 514/3.7; 424/752

(57) ABSTRACT

The invention encompasses methods for enhancing the ability of a companion animal to resist and/or overcome viral infections. The methods of the invention include an amount of lipoic acid that is effective in enhancing the antiviral immunity of a companion animal.

### FIELD OF THE INVENTION

The invention encompasses methods for enhancing the ability of a companion animal to resist and/or overcome viral infections. The methods of the invention include an amount of lipoic acid that is effective in enhancing the antiviral immunity of a companion animal.

### BACKGROUND OF THE INVENTION

Companion animals such as dogs and cats seem to suffer from aging problems. Some of these are manifested in commonplace sayings. One of these is “You can't teach an old dog new tricks.” This saying arises from the observation that as dogs age, their mental capacity seems to diminish as well as physical abilities. Mental activities associated with thinking, learning and memory seem to be lessened (Cummings, B. J., Head, E., Ruehl, W., Milgram, N. W. & Cotman, C. W. (1996): The canine as an animal model of aging and dementia. Neurobiology of Aging 17:259-268). Additionally, behavioral change can be manifested in the aging animals in association with the changing mental capacity. Many causes have been assigned to this lessening of capacity.

These losses in capacity are generally observed in aged canines and felines. Dogs of seven years or older and felines of seven years or older are considered aged and can experience this problem.

The presence of significant levels of at least one antioxidant in the diet of an adult companion pet or fed to a pet outside his diet can inhibit the onset of deterioration of the mental capacity of the aged companion pet and/or maintain the mental capacity of the adult companion pet further into the aged years.

### SUMMARY OF THE INVENTION

In accordance with the present invention, there is provided methods of treating a viral infection in a companion animal including administering to a companion animal in need thereof a food composition comprising an effective amount of one or more antioxidants, for example, lipoic acid.

Another embodiment encompasses methods of enhancing or increasing the immune response to a viral infection in a companion animal comprising administering to a companion animal in need thereof a food composition including an effective amount of one or more antioxidants, for example, lipoic acid.

In all of these methods, it is desirable to administer the antioxidant or mixture thereof in the diet of the animal.

### DETAILED DESCRIPTION OF THE INVENTION

### General Description

The invention encompasses methods of enhancing a companion animal's ability to resist or combat a viral infection comprising administering to a companion animal a food composition comprising an effective amount of lipoic acid to enhance a companion animal's ability to resist or combat a viral infection.

In certain embodiments, the effective amount of lipoic acid to enhance a companion animal's ability to resist or combat a viral infection is at least about 25 ppm.

In certain embodiments, the effective amount is at least about 50 ppm.

In certain embodiments, the effective amount is at least about 100 ppm.

In certain embodiments, the effective amount is about 100 ppm to about 600 ppm.

In certain embodiments, the effective amount is about 100 ppm to about 200 ppm.

In certain embodiments, the companion animal is a dog.

In certain embodiments, the companion animal is a cat.

In certain embodiments, the effective amount is effective to enhance innate antiviral activity in a companion animal.

In certain embodiments, the pet food composition comprising lipoic acid is administered at least 15 days.

In certain embodiments, the pet food composition comprising lipoic acid is administered at least 30 days.

In certain embodiments, the pet food composition comprising lipoic acid is administered at least 45 days.

In certain embodiments, the pet food composition comprising lipoic acid is administered daily.

The invention also encompasses methods of treating a viral infection in a companion animal comprising administering to a companion animal in need thereof a food composition comprising an effective amount of lipoic acid.

In certain embodiments, the effective amount of lipoic acid is at least about 25 ppm.

In certain embodiments, the effective amount is at least about 50 ppm.

In certain embodiments, the effective amount is at least about 100 ppm.

In certain embodiments, the effective amount is about 100 ppm to about 600 ppm.

In certain embodiments, the effective amount is about 100 ppm to about 200 ppm.

In certain embodiments, the companion animal is a dog.

In certain embodiments, the companion animal is a cat.

In certain embodiments, the pet food composition comprising lipoic acid is administered at least 15 days.

In certain embodiments, the pet food composition comprising lipoic acid is administered at least 30 days.

In certain embodiments, the pet food composition comprising lipoic acid is administered at least 45 days.

In certain embodiments, the pet food composition comprising lipoic acid is administered daily.

The diet fed to the adult companion pet, for example canine and feline, is the standard normal diet fed to an animal of that age. Below is a typical diet for a canine of 1 to 6 years of age.

TABLE 1

| Component | Target |
|---|---|
| Protein (% of dry matter) | 23 |
| Fat (% of dry matter) | 15 |
| Phosphorus (% of dry matter) | 0.6 |
| Sodium (% of dry matter) | 0.3 |

The inventors have also surprisingly found that the addition of one or more antioxidants, for example lipoic acid, is useful in enhancing the innate antiviral immune function in companion animals, for example, dogs and cats. As used herein, the term “enhance” or “enhancing” when referring to antiviral immune function refers to the ability of a companion animals to have an increased immune response to an antigen and thereby be more resistant to infection or clear viral infections from the system of the companion animal faster. Accordingly, a companion animal, for example, a dog, eating a pet food containing an antioxidant, for example, lipoic acid will be more resistant to and will clear viral infections faster than an animal not consuming antioxidants.

The component in the diet which accomplishes this is an antioxidant or mixture thereof. An antioxidant is a material that quenches a free radical. Examples of such materials include foods such as ginkgo biloba, citrus pulp, grape pomace, tomato pomace, carrot and spinach, all preferably dried, as well as various other materials such as beta-carotene, selenium, coenzyme Q10 (ubiquinone), lutein, tocotrienols, soy isoflavones, S-adenosylmethionine, gluthathione, taurine, N-acetylcysteine, vitamin E, vitamin C, alpha-lipoic acid, L-carnitine and the like. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The D form is preferable but racemic mixtures are acceptable. The forms and derivatives will function in a Vitamin E like activity after ingestion by the pet. Vitamin C can be administered in this diet as ascorbic acid and its various derivatives thereof such as calcium phosphate salts, cholesteryl salt, 2-monophosphate, and the like, which will function in a vitamin C like activity after ingesting by the pet. They can be in any form such as liquid, semisolid, solid and heat stable form. Alpha-lipoic acid can be administered into the diet as alpha-lipoic acid or as a lipoate derivative as in U.S. Pat. No. 5,621,117, racemic mixtures, salts, esters or amides thereof. L-carnitine can be administered in the diet and various derivatives of carnitine such as the salts such as the hydrochloride, fumarate and succinates, as well as acetylated carnitine and the like, can be used.

The quantities administered in the diet, all as wt % (dry matter basis) of the diet, are calculated as the active material, per se, that is measured as free material. The maximum amounts employed should not bring about toxicity.

At least about 100 ppm or at least about 150 ppm of vitamin E can be used. In certain embodiments, the range of about 500 to about 1,000 ppm can be employed. Although not necessary a maximum of about 2,000 ppm or about 1,500 ppm is generally not exceeded.

With respect to vitamin C at least about 50 ppm is used, desirably at least about 75 ppm and more desirably at least about 100 ppm. A nontoxic maximum can be employed.

The quantity of alpha-lipoic acid can vary from at least about 25, desirably at least about 50 ppm, more desirably about 100 ppm. In various embodiments, the range of lipoic acid that can be administered dogs is about 150 ppm to about 4500 ppm. In various embodiments, the range of lipoic acid that can be administered cats is about 65 ppm to about 2600 ppm. Maximum quantities can vary from about 100 ppm to 600 ppm or to an amount which remains nontoxic to the pet. In certain embodiments, a range is from about 100 ppm to about 200 ppm.

For L-carnitine about 50 ppm, desirably about 200 ppm, more desirably about 300 ppm for canines are a useful minimum. For felines, slightly higher minimums of L-carnitine can be employed such as about 100 ppm, 200 ppm, and 500 ppm. A nontoxic maximum quantity can be employed, for example, less than about 5,000 ppm. For canines, lower quantities can be employed, for example, less than about 5,000 ppm. For canines a preferred range is about 200 ppm to about 400 ppm. For felines a preferred range is about 400 ppm to about 600 ppm.

Beta-carotene at about 1-15 ppm can be employed.

Selenium at about 0.1 up to about 5 ppm can be employed.

Lutein: at least about 5 pm can be employed.

Tocotrienols: at least about 25 ppm can be employed.

Coenzyme Q10: at least about 25 ppm can be employed.

S-adenosylmethionine: at least about 50 ppm can be employed.

Taurine: at least about 1000 ppm can be employed.

Soy isoflavones: at least about 25 ppm can be used.

N-acetylcysteine: at least about 50 ppm can be used.

Glutathione: at least about 50 ppm can be used.

Gingko biloba: at least 50 ppm of extract can be used.

The following are raw ingredients that are high in ORAC (Oxygen radical absorbing capacity) content: Spinach pomace, Tomato pomace, Citrus pulp, Grape pomace, Carrot granules, Broccoli, Green tea, Ginkgo biloba and Corn gluten meal. When added to the diet as 1% inclusions (for a total of 5% substitution for a low ORAC ingredient such as corn) they increased the ORAC content of the overall diet and increased the ORAC content of the plasma of the animals which ate the diet containing these components. Preferably, any ingredient with an ORAC content >25.mu.mole of Trolox equivalents per gram of dry matter could be used if added at 1% in combination with four other 1% ingredients for a total of 5% addition to the diet.

### EXAMPLE 1

Experimental Conditions

Twenty dogs were fed for 30 days. Ten were fed an AAFCO level control food and 10 other dogs were fed the AAFCO level control food containing 150 ppm alpha-lipoic acid. At the end of the end of the 30 days whole blood samples were collected from each dog in Paxgene tubes.

Total RNAs were isolated from whole blood samples using the PAXgene RNA isolation kit. All measurements were done with the canine 2 Affymetrix genechips. For statistical analysis, all measurements were normalized with RMA. All analysis was preformed using Partek. An ANOVA t-test was performed for genes that are differentially expressed between the control and test foods. (at least a 20% change in expression with a pvalue <0.05)

Differentially expressed genes were analyzed with the GeneGo pathway analysis software. Dogs fed lipoic acid for 30 days exhibited an interferon mediated antiviral response. Genes up-regulated by feeding dogs lipoic acid for 30 days that are involved in interferon mediated antiviral response are listed in Table 4.

TABLE 1

| | | | 30-days lipoic | |
|---|---|---|---|---|
| | | | acid canine adult | |
| Gene | | | Fold Up- | p- |
| Symbol | Protein | Protein Name | regulated | value |
| CREBBP | CBP | CREB-binding | 1.2 | 0.04 |
| | Human | protein | | |
| EIF2AK2 | E2AK2 | Interferon- | 1.4 | 0.04 |
| | Human | induced double | | |
| | | stranded RNA- | | |
| | | activated protein | | |
| IFNAR2 | INAR2 | interferon- | 1.3 | 0.01 |
| | Human | alpha/beta | | |
| | | receptor beta | | |
| | | chain precursor | | |
| IFNGR2 | INGR2 | interferon- | 1.2 | 0.03 |
| | Human | gamma receptor | | |
| | | beta chain | | |
| | | precursor | | |
| IRF9 | IRF9 | interferon | 1.3 | 0.2 |
| | Human | regulatory factor | | |
| | | 9 | | |
| JAK2 | JAK2 | Tyrosine protein | 1.4 | 0.01 |
| | Human | kinase JAK2 | | |
| RNASEL | RN5A | 2-5A-dependent | 1.5 | 0.04 |
| | Human | ribonuclease | | |

Based on the studies of dogs fed lipoic acid for 30 days, the inventors have surprisingly found that cell surface receptors for interferon alpha/beta and interferon gamma are increased leading to the potential for increasing the entire interferon mediated antiviral defense mechanism. The inventors have found that JAK2, a key activator of STAT1 and STAT2, is up regulated. Interferon regulatory factor 9 (IFR9) is up-regulated. IFR9, STAT1 and STAT2 form a complex (ISFG3) that translocates to the nucleus and up regulates the antiviral genes, interferon-induced, double stranded RNA-activated protein kinase (PKR) and 2-5A-dependent ribonuclease (RnaseL). PKR inhibits elF2S1 via phosphorylation leading to an inhibition of viral protein synthesis. RnaseL cleaves viral RNA inhibiting viral replication and function.

The invention is not to be limited in scope by the specific embodiments disclosed in the examples, which are intended as illustrations of a few aspects of the invention, and any embodiments, which are functionally equivalent, are within the scope of this invention. Indeed, various modifications of the invention in addition to those shown and described herein will become apparent to those skilled in the art and are intended to fall within the appended claims.

For any references that have been cited, the entire disclosures of which are incorporated herein by reference.

What is claimed is:

1. A method of enhancing a companion animal's ability to resist or combat a viral infection comprising administering to a companion animal in need thereof a food composition comprising an effective amount of lipoic acid and one or more of vitamin E, vitamin C, beta-carotene, selenium, lutein, tocotrienols, coenzyme Q10, S-adenosylmethioine, taurin, soy isoflavones, N-acetylcysteine, glutathione, and gingko biloba to enhance a companion animal's ability to resist or combat a viral infection,
wherein said effective amount of lipoic acid to enhance a companion animal's ability to resist or combat a viral infection is at least about 25 ppm.

2. The method of claim 1, wherein the effective amount is at least about 50 ppm.

3. The method of claim 1, wherein the effective amount is at least about 100 ppm.

4. The method of claim 1, wherein the effective amount is about 100 ppm to about 600 ppm.

5. The method of claim 1, wherein the effective amount is about 100 ppm to about 200 ppm.

6. The method of claim 1, wherein the companion animal is a dog.

7. The method of claim 1, wherein the companion animal is a cat.

8. The method of claim 1, wherein the effective amount is effective to enhance innate antiviral activity in a companion animal.

9. The method of claim 1, wherein the pet food composition comprising lipoic acid is administered at least 15 days.

10. The method of claim 1, wherein the pet food composition comprising lipoic acid is administered at least 30 days.

11. The method of claim 1, wherein the pet food composition comprising lipoic acid is administered at least 45 days.

12. The method of claim 1, wherein the pet food composition comprising lipoic acid is administered daily.

13. A method of treating a viral infection in a companion animal comprising administering to a companion animal in need thereof a food composition comprising an effective amount of lipoic acid, and one or more of vitamin E, vitamin C, beta-carotene, selenium, lutein, tocotrienols, coenzyme Q10, S-adenosylmethioine, taurin, soy isoflavones, N-acetylcysteine, glutathione, and gingko biloba,
wherein said effective amount of lipoic acid is at least about 25 ppm.

14. The method of claim 13, wherein the effective amount is at least about 50 ppm.

15. The method of claim 13, wherein the effective amount is at least about 100 ppm.

16. The method of claim 13, wherein the effective amount is about 100 ppm to about 600 ppm.

17. The method of claim 13, wherein the effective amount is about 100 ppm to about 200 ppm.

18. The method of claim 13, wherein the companion animal is a dog.

19. The method of claim 13, wherein the companion animal is a cat.

20. The method of claim 13, wherein the pet food composition comprising lipoic acid is administered at least 15 days.

21. The method of claim 13, wherein the pet food composition comprising lipoic acid is administered at least 30 days.

22. The method of claim 13, wherein the pet food composition comprising lipoic acid is administered at least 45 days.

23. The method of claim 13, wherein the pet food composition comprising lipoic acid is administered daily.

24. The method of claim 1, wherein the pet food composition induces an interferon-mediated antiviral response.

25. The method of claim 13, wherein the pet food composition induces an interferon-mediated antiviral response.
